(19) United States
(12) Patent Application Publication
(10) Pub. No.: US 2019/0314077 A1
(43) Pub. Date: Oct. 17, 2019

(54) CATHETER-BASED ABLATION SYSTEMS AND METHODS OF ABLATION

(71) Applicants: Mehdi Razavi, Houston, TX (US); Elaine Chinn, Fate, TX (US); Charlene Morrison, McKinney, TX (US); Ryan Oatman, Conneaut, OH (US); Ashley Rook, Kingwood, TX (US); Blake Smith, Baytown, TX (US); Anand Ganapathy, Los Angeles, CA (US); Mathews John, Houston, TX (US); David Burkland, Houston, TX (US); Brian Greet, Houston, TX (US)

(72) Inventors: Mehdi Razavi, Houston, TX (US); Elaine Chinn, Fate, TX (US); Charlene Morrison, McKinney, TX (US); Ryan Oatman, Conneaut, OH (US); Ashley Rook, Kingwood, TX (US); Blake Smith, Baytown, TX (US); Anand Ganapathy, Los Angeles, CA (US); Mathews John, Houston, TX (US); David Burkland, Houston, TX (US); Brian Greet, Houston, TX (US)

(21) Appl. No.: 16/381,824

(22) Filed: Apr. 11, 2019

Related U.S. Application Data
(60) Provisional application No. 62/657,262, filed on Apr. 13, 2018.

(51) Int. Cl. A61B 18/04

(52) U.S. Cl. CPC A61B 18/04; A61B 2018/00261

(57) ABSTRACT

Catheter ablation systems are used to isolate the Left Atrial Appendage (“LAA”), or portions of the LAA, by using balloons. The systems deliver an ablation fluid such as alcohol in order to destroy the LAA tissue isolated between the balloons, deliver saline to dilute the ablation fluid, and remove excess fluid and particulates by suction to prevent excess residual alcohol from remaining in the LAA.

### CROSS-REFERENCE TO RELATED APPLICATIONS

This application claims priority to U.S. provisional application Ser. No. 62/657,262 filed on Apr. 13, 2018, the content of which is incorporated herein by reference.

### BACKGROUND

This disclosure relates generally to catheter-based ablation systems and methods of using catheter-based ablation systems. More specifically, this disclosure relates to endovascular catheter systems for ablation of the Left Atrial Appendage (“LAA”) or other cardiovascular tissue, and methods of ablation of the LAA using an ablation fluid, such as alcohol.

Atrial Fibrillation (“AFib”) is an arrhythmia characterized by a rapid and irregular heart rate. Atrial fibrillation occurs when faulty electrical signals disrupt the normal timing of the heart's inherent pacemaker, which can cause the atria to quiver and the ventricles to beat faster. When the atria quivers instead of contracting rhythmically, blood can pool in the atria, which increases the risk of clot formation and stroke. Excessive or irregular heartbeats can overwork the heart muscle and lead to heart failure.

An estimated 2.7 to 6.1 million people in the United States suffer from AFib. Older adults contribute largely to this number, with 70% of people with AFib being between the ages of 65 and 85. Furthermore, men have a higher incidence of AFib than women. People affected by atrial fibrillation are 5 to 7 times more likely to experience a stroke, which is the fifth leading cause of death in the United States, killing nearly 130,000 people a year.

One region of the heart in particular, the LAA, is especially at risk of creating blood clots. The LAA is a sac in the muscle wall of the left atrium; blood often pools in this sac, creating the possibility for clot formation and subsequent stroke. Additionally, the LAA tissue can propagate the faulty electrical signals to the remainder of the heart, worsening the effects of the arrhythmia.

Current methods for treatment of patients with AFib include managing the risk of clotting and stroke, which can involve anticoagulant medications or mechanical isolation of the LAA. For example, the WATCHMAN implant is designed to close off the left atrial appendage to prevent the flow of blood into the appendage and reduce the formation of clots. The WATCHMAN implant is made up of a frame with mesh covering that expands to fit the size of the left atrial opening. Alternatively, percutaneous left atrial appendage suture ligation can be performed using the LARIAT device. The LARIAT procedure introduces a mechanism by which the left atrial appendage can be excluded in the absence of an implantable device. Neither anticoagulant medications nor mechanical isolation of the LAA electrically isolates the dysrhythmic cardiac tissue, so they are not curative.

Ablation of dysrhythmic cardiac tissue is also an option, but current ablation procedures are more difficult as a result of the geometry of the LAA, and there is no guarantee that all of the diseased tissue will be destroyed as the LAA is often a source of aberrant signals in atrial fibrillation. Examples of development efforts in ablation technology have been described in U.S. Pat. Appl. Pub. No. 2018/0000314(A1), entitled “Methods and apparatus for treatment of atrial fibrillation,” U.S. Pat. Appl. Pub. No. 2002/0087151(A1), entitled “Tissue ablation apparatus with a sliding ablation instrument and method,” U.S. Pat. Appl. Pub. No. 2012/0143177(A1), entitled “Catheter systems for cardiac arrhythmia ablation,” U.S. Pat. Appl. Pub. No. 2005/0228468(A1), entitled “Devices, systems, and methods for treating atrial fibrillation,” and U.S. Pat. Appl. Pub. No. 2016/0066991(A1), entitled “Methods and systems for accessing a pericardial space and preventing strokes arising from left atrial appendage.” Ablation of dysrhythmic cardiac tissue may be performed using a catheter. Examples of development efforts in catheter technology have been described in U.S. Pat. Appl. Pub. No. 2012/0245574(A1), entitled “Spray nozzle design for a catheter,” U.S. Pat. No. 5,324,269, entitled “Fully exchangeable dual lumen over-the-wire dilatation catheter with rip seam,” U.S. Pat. No. 5,919,163, entitled “Catheter with slidable balloon,” and U.S. Pat. No. 5,318,535, entitled “Low-profile dual-lumen perfusion balloon catheter with axially movable inner guide sheath.”

Despite these efforts, there is a continued need for ablation systems suitable for the ablation of dysrhythmic cardiac tissue, in particular, the LAA.

### BRIEF SUMMARY OF THE DISCLOSURE

In some aspects, the disclosure describes an endovascular catheter system. The endovascular catheter system may be used for ablation of the LAA or other cardiovascular tissue.

The endovascular catheter system may comprise an outer catheter. The outer catheter may include a proximal open end, a distal open end, and an internal lumen. The endovascular catheter system may comprise a first balloon fixed to the outside of the outer catheter. The first balloon may be located proximate to the distal open end of the outer catheter. The endovascular catheter system may comprise a first inflation tube connecting a proximal end of the outer catheter and the first balloon. The first inflation tube may be located inside a wall of the outer catheter.

The endovascular catheter system may comprise a multi-lumen body. The multi-lumen body may include a first lumen. The first lumen may be used for delivery of fluid, for example, under pressure. The first lumen may have a proximal open end and a distal closed end. The first lumen may include spray openings disposed proximate to the distal closed end of the first lumen. The multi-lumen body may also include a second lumen. The second lumen may be used for suctioning fluid, for example, under vacuum. The second lumen may have a proximal open end and a distal closed end. The second lumen may be located inside the first lumen so that the second lumen has a diameter equal to a fraction of a diameter of the first lumen. The second lumen may extend beyond the distal closed end of the first lumen. The second lumen may include suction openings disposed beyond the distal closed end of the first lumen toward the distal closed end of the second lumen.

In some embodiments, the multi-lumen body may be integral to the outer catheter. The first balloon may be located before the spray openings and the suction openings in the multi-lumen body toward the proximal open end of the first lumen and the second lumen.

In some embodiments, the endovascular catheter system may comprise an inner catheter. The multi-lumen body may be formed in the inner catheter. The inner catheter may include a proximal open end and a closed distal end. The inner catheter may have an outer diameter that is smaller than a diameter of the internal lumen of the outer catheter. The inner catheter may further include a second balloon fixed to the outside of the inner catheter beyond the suction openings toward the closed distal end of the inner catheter. The inner catheter may further include a second inflation tube connecting the proximal open end of the inner catheter and the second balloon. The second inflation tube may be located inside a wall of the inner catheter. In use, the closed distal end of the inner catheter, the spray openings and the suction openings in the multi-lumen body, can be extended beyond the distal open end of the outer catheter. The second balloon may be movable relative to the first balloon so that an ablation window of variable length can be created.

The endovascular catheter system may further comprise a sensor exposed to fluid in the second lumen. The sensor may include one or more of an alcohol sensor, an optical sensor, and a pressure sensor. Alternatively or additionally, the endovascular catheter system may further comprise a sensor located proximate to the spray openings and the suction openings. The sensor may include one or more of an alcohol sensor, an optical sensor, and a pressure sensor. The endovascular catheter system may comprise radiopaque bands.

In some aspects, the disclosure describes a method of ablation of the LAA. The method may comprise the step of introducing the outer catheter into the left atrium along a guide wire and positioning the outer catheter at the neck of the LAA. The method may comprise the step of inflating the first balloon attached to the outer catheter to seal against a wall of the LAA. The method may comprise the step of suctioning blood via the suction openings connected to the first lumen. The method may comprise the step of delivering an ablation fluid via the spray openings connected to the second lumen to coat the walls of the LAA. The ablation fluid may be alcohol. The method may further comprise incorporating a radiopaque dye or contrast into the ablation fluid. The method may comprise the step of delivering saline via the spray openings while simultaneously suctioning the ablation fluid and particulates via the suction openings. The method may further comprise measuring an ablation fluid dilution during the step of delivering saline. The method may comprise the step of deflating the first balloon.

In some embodiments, the method may further comprise the step of advancing the inner catheter into which the first lumen and the second lumen are formed through the outer catheter. The method may further comprise the step of extending a distal tip of the inner catheter into the LAA beyond the first balloon. The method may further comprise the step of inflating the second balloon attached to the inner catheter to seal against the wall of the LAA. The method may comprise the step of isolating an ablation window between the first balloon and the second balloon.

### BRIEF DESCRIPTION OF THE DRAWINGS

For a more detailed description of the embodiments of the disclosure, reference will now be made to the accompanying drawings, wherein:

FIG. 1 shows an outer catheter, useable for isolating the LAA from the rest of the heart with a first balloon and for housing an inner catheter;

FIG. 2 shows an inner catheter, useable for ablation and removal of ablation material with spraying and suctioning mechanisms, as well as a second balloon useable to create an isolated ablation window;

FIG. 3A shows a cross-section view of the outer catheter shown in FIG. 1;

FIG. 3B shows a cross-section view of the inner catheter shown in FIG. 2; and

FIG. 4 shows a catheter system and its ablation window within the LAA.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention.

All numerical values in this disclosure may be approximate values unless otherwise specifically stated. Accordingly, various embodiments of the disclosure may deviate from the numbers, values, and ranges disclosed herein without departing from the intended scope.

The exemplary embodiments presented below may be combined in any combination of ways, i.e., any element from one exemplary embodiment may be used in any other exemplary embodiment, without departing from the scope of the disclosure.

This disclosure describes examples of methods for ablating the tissue of the LAA specifically in any capacity. The methods can utilize alcohol for intra-cardiac ablation. The methods involve an endovascular catheter that can provide simultaneous delivery of alcohol and suction of the alcohol. The methods may result in electrically isolating the LAA rather than only physically isolating it, as well as removing continued hormone secretion from the LAA. The methods may be less invasive than suture ligation while accomplishing the same goal.

This disclosure also describes examples of endovascular, intra-cardiac, catheter ablation systems capable of isolating the LAA. These systems may provide operators maximal control to ablate tissue: either ablate a small portion of the tissue of the LAA or ablate the entire LAA. These systems can also be used in other portions of the cardiovascular system to provide controlled ablation.

The endovascular, intra-cardiac, catheter ablation systems are capable of isolating the LAA, or portions of the LAA, by, for example, using a two-balloon system, delivering alcohol in order to destroy the isolated tissue, delivering saline to dilute the delivered alcohol, and suctioning to remove excess fluid and particulates and prevent excess residual alcohol from remaining in the body. One or more sensors can be included in the suction port tubing or distal catheter tip to sense the levels of alcohol to ensure no residual alcohol remains in the body of the patient.

The endovascular, intra-cardiac, catheter ablation systems can alternatively be used to deliver another fluid or chemical as the ablation material. Furthermore, alcohol or other ablation fluids can be delivered in alternative ways, including an alcohol coated balloon, a brush coated/dipped in alcohol to apply alcohol to the walls of the LAA, or an alcohol gel.

In some examples, the catheter ablation systems may consist of only one integrated catheter. In other examples, the catheter ablation system may comprise a plurality of catheters (an example system described herein comprises two separate catheters).

The catheter(s) may include only one proximal balloon to allow an entire cardiac chamber to be sprayed with alcohol or may include two or more balloons. Sensors to detect alcohol can utilize traditional alcohol sensors, color sensors using colored alcohol, pH sensors, or other relevant sensors.

The endovascular, intra-cardiac, catheter ablation systems provide a therapeutic option for patients with AFib and potentially heart failure. These systems may be used in situations where controlled ablation of a cardiac chamber (most commonly the LAA) is required. Given that the window of ablation is achieved through a two-balloon system, these systems can be used in controlled ablation of any region of the cardiovascular system by an endovascular means.

The catheter ablation system illustrated in FIGS. 1-4 comprises an outer catheter 10 and an inner catheter 20. A window of operation, the ablation window 102, is created by balloons 18 and 28 provided at the respective ends of each catheter. Fluids, including alcohol (ethanol most commonly), saline, and contrast can be delivered into and suctioned from the ablation window 102.

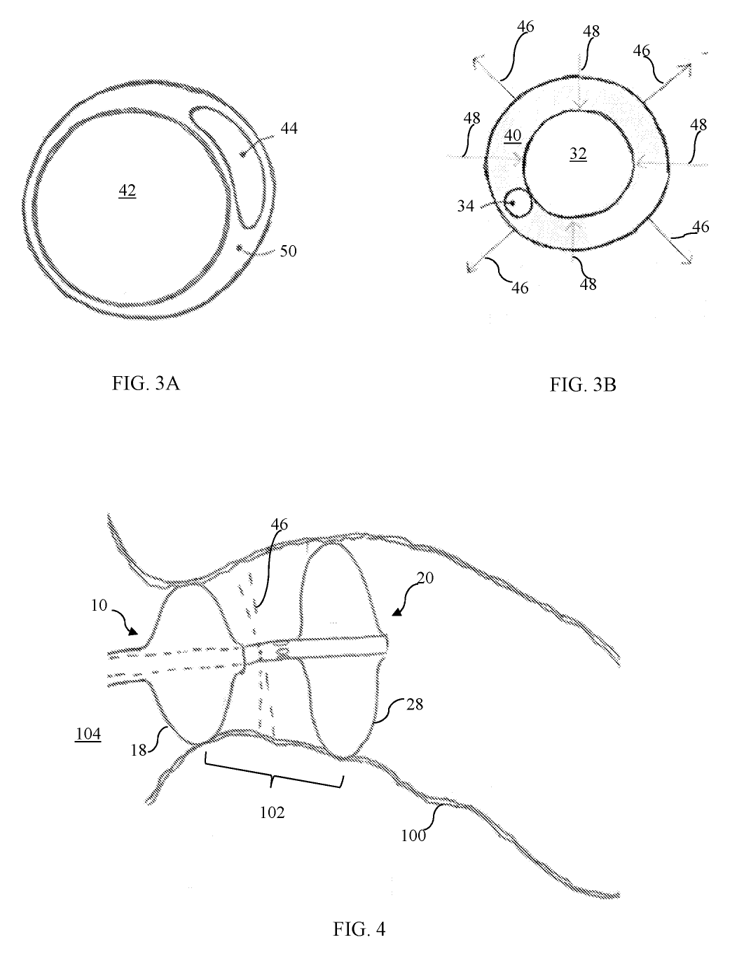

FIGS. 1 and 3A show the outer catheter 10 used to isolate the LAA 100 from the rest of the heart and to house the inner catheter 20. Outer catheter 10 has a proximal end with an opening 12 for the insertion of inner catheter 20 and a port 14 for the inflation of a first balloon 18. The outer catheter 10 has a distal end with a first balloon 18 attached to it. There are optional radiopaque bands 16 located on the outer catheter 10 just before and just after the first balloon 18, for example, to aid in imaging during the procedure.

An inner lumen 42 is formed in the body 50 of outer catheter 10. The inner lumen 42 is open at both ends for receiving a portion of the inner catheter 20, and an inflation tube 44 (or secondary lumen) for the inflation of the first balloon 18.

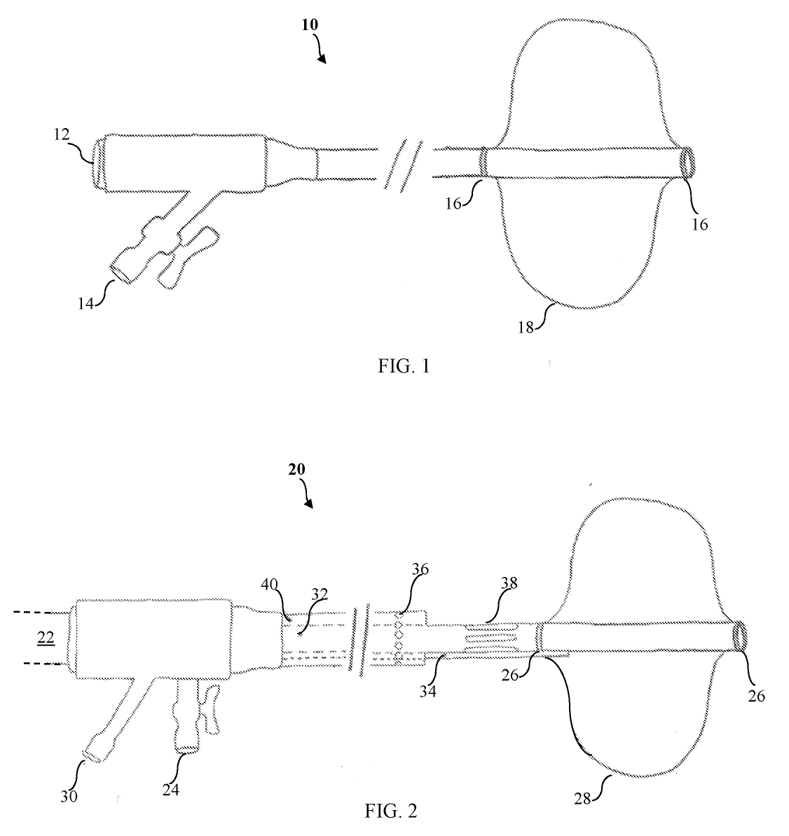

FIGS. 2 and 3B show the inner catheter 20 used for delivery of ablation fluid and removal of ablation material. The inner catheter 20 has a proximal end with a port 30 for alcohol/saline, a port 24 for balloon inflation fluid, and a suction port 22, and a distal end with the second balloon 28 attached to it. The second balloon 28 is used to create or isolate the ablation window 102. The inner catheter 20 is provided with spraying mechanism 46 and suctioning mechanism 48 and is preferably able to perform both spraying and suction functions simultaneously. As illustrated in FIG. 4, the second balloon 28, the spraying mechanism 46, and the suctioning mechanism 48 are intended for use in the LAA 100 past the location of first balloon 18 of the outer catheter 10. Multiple lumens 32, 40 are formed in the inner catheter 20.

The outermost lumen 40 of inner catheter 20 is preferably used for delivery of fluid, including alcohol, saline, and contrast. The outermost lumen 40 has an outer diameter that may equal to that of a 12 F catheter and is less than the inner diameter of the inner lumen 42 of the outer catheter 10. The outermost lumen 40 has an inner diameter that is some fraction of its outer diameter. The outermost lumen 40 does not span the entirety of the length of inner catheter 20 and ends some distance before the second balloon 28. At the distal end of the outermost lumen 40 are circumferential holes 36 for the delivery of fluid under pressure.

The inner lumen 32 of inner catheter 20 is preferably used for suctioning fluid under a vacuum. The inner lumen 32 extends beyond the outermost lumen 40. The second balloon 28 is attached to the end of the inner lumen 32. There are circumferential holes 38 across the inner lumen 32 between the end of the outermost lumen 40 and the start of the second balloon 28, into which fluid may be removed by suction. Along the inner lumen 32 is a smaller inflation tube 34 (or tertiary lumen) connected to the second balloon 28 for the inflation fluid (usually air). There are optional radiopaque bands 26 located just before and just after second balloon 28.

FIG. 4 shows the catheter ablation system and the ablation window 102 within the LAA 100. The positioning of the inner catheter 20 relative to the first balloon 18 and the optional inflation of the second balloon 28 on the inner catheter 20 permit an operator to control the size of the ablation window 102, so that either a small portion of the tissue of the LAA 100, or the entire LAA 100, may be ablated. The ablation window length may be changed by advancing the inner catheter 20 further into the LAA 100. After the inner catheter 20 is inserted into the outer catheter 10 and advanced beyond the first balloon 18, the second balloon 28 is inflated. The two inflated balloons create the ablation window 102. The ablation window 102 also isolates a section of the catheter ablation system that contains the spraying mechanism 46 and suctioning mechanism 48 of the inner catheter 20. A radiopaque band may be provided next to the spraying mechanism 46 on the inner catheter 20 to allow the operator ensuring that the spraying mechanism 46 is beyond the distal end of the outer catheter 10 and to aid the operator when adjusting the length of the ablation window 102.

Preferably, the catheter ablation system ensures complete occlusion of the LAA 100 from the atrium 104 of the heart to avoid leakage of alcohol in the atrium 104. Also, the catheter ablation system is preferably paired with an available device (e.g., WATCHMAN) to ensure complete (electrical, chemical, and mechanical) isolation.

A method of using the catheter ablation system to ablate tissue in the LAA can comprise the following steps:
a. introduction of the outer catheter 10 into the left atrium 104 along a guide wire using established endovascular and trans-septal techniques;
b. location of the LAA 100 using established techniques (pigtail catheter and contrast) and positioning of the outer catheter 10 at the neck of the LAA 100;
c. inflation of the first balloon 18 and verification of proximal seal;
d. advancement of the inner catheter 20 through the outer catheter 10 to desired distance into the LAA 100;
e. inflation of the second balloon 28 and verification of both seals (i.e., the proximal seal created with first balloon 18 and the distal seal created with second balloon 28);
f. suctioning of as much blood as possible from the ablation window 102;
g. delivery of alcohol (ethanol) via the spraying mechanism 46 of the inner catheter 20 to coat the walls of the LAA 100 within the ablation window 102;
h. delivery of saline via the spraying mechanism 46 of the inner catheter 20 with continuous suction via the suction mechanism 48 to dilute and remove the alcohol and any particulates;
i. verification of sufficient alcohol dilution and removal (either through continuous dilution with substantial saline or using a sensor);
j. deflation of the second balloon 28 and removal of the inner catheter 20; and
k. deflation of the first balloon 18 and removal of the outer catheter 10.

Accordingly, the catheter ablation system is used to destroy the LAA tissue in order to electrically isolate the LAA from the rest of the heart, thus preventing the propagation of aberrant electrical activity that contributes to cardiac arrhythmias. This destruction of tissue could, in turn, decrease the likelihood of clot formation and stroke, as particularly seen in AFib. The destruction of tissue could also prevent the release of hormones from the LAA that are thought to contribute to heart failure.

With both the first balloon 18 and the second balloon 28 deployed, a controllable circumferential area of the LAA 100 is ablated. The extent of LAA tissue destroyed is tunable by lengthening or shortening the ablation window 102 and is at the discretion of the operator. Alternatively, with only the first balloon 18 deployed, the entire LAA 100 can be ablated.

The catheter ablation system is preferably used in conjunction with a mechanical LAA closure device, such as the WATCHMAN.

Other embodiments of catheter ablation systems can deliver alcohol by other means. For example, alcohol can be pre-coated around a balloon, presoaked in a swab/brush that can be used to coat a heart chamber, or as a single spray to soak an entire heart chamber.

Variations of the catheter ablation system illustrated in FIGS. 1-4 could involve one or more of the following modifications: sealing the space between catheter 2 and catheter 1, making the system a one-catheter system, using an ablation material other than alcohol, incorporating pressure sensors to monitor the pressure in the LAA during the fluid delivery and suctioning, incorporating a radiopaque dye or contrast into the alcohol or other ablation fluid in order to visualize relative concentration, measuring the concentration of the suctioned fluid in order to verify a safe level of dilution or removal, and making the catheter tips pre-formed into a curve. The catheter ablation system illustrated in FIGS. 1-4 is further susceptible to various modifications and alternative forms known to those having ordinary skill in the art.

While specific embodiments are shown by way of example in the drawings and description, it should be understood, however, that the drawings and detailed description thereto are not intended to limit the claims to the particular form disclosed, but on the contrary, the intention is to cover all modifications, equivalents, and alternatives falling within the scope of the claims.

What is claimed is:

1. An endovascular catheter system for ablation of the left atrial appendage (LAA) or other cardiovascular tissue, comprising:
an outer catheter;
a multi-lumen body, the multi-lumen body including:
a first lumen for delivery of fluid under pressure, the first lumen having a proximal open end and a distal closed end, the first lumen including spray openings disposed proximate to the distal closed end of the first lumen, and
a second lumen for suctioning fluid under vacuum, the second lumen having a proximal open end and a distal closed end, the second lumen being located inside the first lumen so that the second lumen has a diameter equal to a fraction of a diameter of the first lumen, the second lumen extending beyond the distal closed end of the first lumen, the second lumen including suction openings disposed beyond the distal closed end of the first lumen toward the distal closed end of the second lumen;
a first balloon fixed to the outside of the outer catheter; and
a first inflation tube connecting a proximal end of the outer catheter and the first balloon.

2. The endovascular catheter system of claim 1,
wherein the outer catheter includes a proximal open end, a distal open end, and an internal lumen,
wherein the first inflation tube is located inside a wall of the outer catheter,
wherein the first balloon is located proximate to the distal open end of the outer catheter; and
wherein the endovascular catheter system further comprises an inner catheter, including a proximal open end, and a closed distal end,
wherein the inner catheter has an outer diameter that is smaller than a diameter of the internal lumen of the outer catheter,
wherein the multi-lumen body is formed in the inner catheter, and
wherein, in use, the closed distal end of the inner catheter, the spray openings and the suction openings in the multi-lumen body, can be extended beyond the distal open end of the outer catheter.

3. The endovascular catheter system of claim 2, wherein the inner catheter further includes:
a second balloon fixed to the outside of the inner catheter beyond the suction openings toward the closed distal end of the inner catheter; and
a second inflation tube connecting the proximal open end of the inner catheter and the second balloon, wherein the second inflation tube is located inside a wall of the inner catheter.

4. The endovascular catheter system of claim 3, wherein the first balloon is movable relative to the second balloon, so that an ablation window of variable length can be created.

5. The endovascular catheter system of claim 1, wherein the multi-lumen body is integral to the outer catheter.

6. The endovascular catheter system of claim 5, wherein the first balloon is located before the spray openings and the suction openings in the multi-lumen body toward the proximal open end of the first lumen and the second lumen.

7. The endovascular catheter system of claim 1, further comprising a sensor exposed to fluid in the second lumen.

8. The endovascular catheter system of claim 7, wherein the sensor includes one or more of an alcohol sensor, an optical sensor, and a pressure sensor.

9. The endovascular catheter system of claim 1, further comprising a sensor located proximate to the spray openings and the suction openings.

10. The endovascular catheter system of claim 9, wherein the sensor includes one or more of an alcohol sensor, an optical sensor, and a pressure sensor.

11. A method for ablation of the left atrial appendage (LAA) comprising the steps of:
introducing an outer catheter into the left atrium along a guide wire;
positioning the outer catheter at a neck of the LAA;
inflating a first balloon attached to the outer catheter to seal against a wall of the LAA;
suctioning blood via suction openings connected to a first lumen;
delivering an ablation fluid via spray openings connected to a second lumen to coat the walls of the LAA;
delivering saline via the spray openings while simultaneously suctioning the ablation fluid and particulates via the suction openings; and
deflating the first balloon.

12. The method of claim 11 wherein the ablation fluid is alcohol.

13. The method of claim 11 further comprising incorporating a radiopaque dye or contrast into the ablation fluid.

14. The method of claim 11 further comprising measuring an ablation fluid dilution during the step of delivering saline.

15. The method of claim 11 further comprising:
advancing an inner catheter through the outer catheter and extending a distal tip of the inner catheter into the LAA beyond the first balloon, wherein the first lumen and the second lumen are formed into the inner catheter;
inflating a second balloon attached to the inner catheter to seal against the wall of the LAA; and
isolating an ablation window between the first balloon and the second balloon.
